(19) United States
(12) Patent Application Publication
(10) Pub. No.: US 2023/0157848 A1
(43) Pub. Date: May 25, 2023

(54) ORTHOPEDIC DEVICE

(71) Applicant: OTTO BOCK HEALTHCARE PRODUCTS GMBH, Wien (AT)

(72) Inventors: Luis SAGMEISTER, Wien (AT); Christoph LEDINGER, Wien (AT); Florian EDER, Wien (AT)

(21) Appl. No.: 18/056,934

(22) Filed: Nov. 18, 2022

(30) Foreign Application Priority Data
Nov. 23, 2021 (DE) 10 2021 130 657.4

(51) Int. Cl. A61F 2/58; A61F 2/70

(52) U.S. Cl. CPC A61F 2/586; A61F 2/70; A61F 2002/5038

(57) ABSTRACT

An orthopedic device has a base and a pivot element which is mounted in an articulated manner on the base. The pivot element is displaceable via a force transmission element connected to a drive from a starting position into a pivoting position that is pivoted in relation to the starting position. The force transmission element permits passive pivoting of the pivot element in the direction of the pivoting position without activation of the drive. A spring element designed for counteracting passive pivoting of the pivot element into the pivoting position is assigned to the pivot element. The spring element is formed separately from the force transmission element, and the force transmission element blocks deformation of the spring element during pivoting by the drive into the respective pivoting position.

### FIELD OF THE INVENTION

The invention relates to an orthopedic device with a base and a pivot element which is mounted in an articulated manner on the base and is displaceable via a force transmission element, which is connected to a drive, from a starting position into a pivoting position that is pivoted in relation to the starting position, wherein the force transmission element permits passive pivoting of the pivot element in the direction of the pivoting position without activation of the drive. In particular, such an orthopedic device is in the form of a prosthesis or a prosthesis component of the upper extremity, in particular in the form of part of a prosthetic hand. In principle, it is also possible for the orthopedic device to be in the form of an orthosis or exoskeleton.

### BACKGROUND

Driven orthopedic devices can permit active support, i.e. displacement of two components with respect to each other via a motorized drive, in one direction only, whereas a passive return takes place in the opposite direction, i.e. a return by relaxation of a spring element or of another energy store. Alternatively, for a return or a reversal of movement, for example in opposite pivoting directions or movement directions, use is made of drives which each work in the opposite direction, or an individual drive with a rigid coupling is activated in the opposite direction of movement.

Specifically in the case of a gripping device or holding device, for example a prosthetic hand or an orthotic hand, it is helpful if the drive is self-locking and a relative displacement can no longer take place when the drive is switched off, for example if an external force acts on the driven element of the orthopedic device.

A hand prosthesis with a chassis, on which at least one prosthetic finger is mounted in an articulated manner and which is movable about at least one pivot axis via a drive which is connected to the prosthetic finger via a force transmission device, is known from DE 10 2005 061 266 A1. The force transmission device between the drive and the prosthetic finger is rigid under tension and yielding under pressure and elastic under bending and resiliently elastic, with the spring rate of the force transmission device being dimensioned in such a manner that, when the force transmission device is subjected to a compressive force, the finger prosthesis returns into a starting position. The force transmission device can be in the form of a spring-damper unit or in the form of a spring which is optionally pretensioned.

### SUMMARY

It is the object of the present invention to provide an orthopedic device which can more easily be adapted to the individual circumstances of the user.

According to the invention, this object is achieved by an orthopedic device having the features described in the description and the figures.

The orthopedic device with a base and a pivot element which is mounted in an articulated manner on the base and is displaceable via a force transmission element, which is connected to a drive, from a starting position into a pivoting position that is pivoted in relation to the starting position, wherein the force transmission element permits passive pivoting of the pivot element in the direction of the pivoting position without activation of the drive, and a spring element designed for counteracting passive pivoting of the pivot element into the pivoting position is assigned to the pivot element makes provision for the spring element to be formed separately from the force transmission element, and for the force transmission element to block deformation of the spring element during pivoting by the drive into the respective pivoting position. In the case of the conventional driven orthopedic devices which permit passive adjustment and flexibility of the pivot element to be pivoted in the event of actions of external forces on the other side of the drive, a resilient mounting of the pivot element is provided, in which the spring element is generally tensioned via the drive when the drive is activated. This leads to a delayed transmission of force and to an increased consumption of energy. Furthermore, the positioning accuracy is limited. According to the invention, provision is made for the spring element not to be connected in series with the force transmission element during pivoting by the drive, and therefore the force to be transmitted to the pivot element is not conducted through the spring element. Instead, the force transmission element transmits the tensile force or compressive force, depending on the direction of force, to the pivot element independently of the spring element. The spring element ensures that the force transmission element is under pretension in relation to the pivot element, and therefore, when the drive is activated, force can be transmitted directly in the force transmission direction and therefore the pivot element can be precisely displaced. Furthermore, the spring element permits displacement of the pivot element if a force acts on the orthopedic device from the outside. The separate embodiment of the spring element with respect to the force transmission element makes it possible for the spring element to be able to be exchanged without the force transmission element having to be replaced at the same time. As a result, it is possible to adapt the respective spring element to the requirements or desires of the respective user of the orthopedic device and in particular to use identical force transmission elements if a plurality of pivot elements are provided on a base or for different models. This reduces the number of parts which have to be kept in store and increases the degree of individualization.

The force transmission element and spring element are jointly displaced by the drive, for example, whenever there is no passive pivoting of the pivot element by an external force and therefore the pivot element is pretensioned against the force transmission element by the spring element. In the event of passive pivoting of the pivot element without activation of the drive, the force transmission element can remain unmoved or can be moved at the same time.

The spring element can be mounted directly on the force transmission element or on an abutment which is displaceable together with the force transmission element, and therefore the spring element is moved together with the force transmission element into the pivoting position via the drive. Furthermore, the spring element is supported on the pivot element or acts on the pivot element in such a manner that it is in a pretensioned position in relation to the force transmission element. The force transmission element is under tension by means of the spring element if no external forces act on the pivot element. In the case of a partial compressive-force-transmitting embodiment of the force transmission element or in the case of a compressive-force-transmitting embodiment of the abutment, it is possible to bring about the return movement of the pivot element by a reversal of movement of the drive. If the abutment is moved in the direction of the pivot element, compressive forces are transmitted via the spring element to the pivot element such that the latter is moved into an open position, for example for opening a prosthetic hand. The return movement from the pivoting position into the starting position then takes place via the compressive force of the spring element which is moved together with the abutment or with a compressive-force-transmitting section of the force transmission element.

The force transmission element can be rigid under tension and yielding under pressure, for example can be in the form of a flexible tension means, for example a rope, cable, strap, chain or belt, or in the form of a combination of a plurality of flexible tension means. Alternatively, the force transmission element can be in the form of a movably mounted rod, a movably mounted sleeve or in the form of a telescopic rod. A movably mounted rod or sleeve permits transmission of tensile force when a drive is activated and, when a drive is at a standstill, passive displacement counter to the spring force of the spring element by one end of the rod or sleeve being mounted, for example, in a sliding guide, the sliding guide having a stop in order to block displacement in the force transmission direction. A corresponding manner of operation is achieved with a telescopic rod which has a predetermined maximum length and can be pushed together when an external force is exerted on the pivot element. The telescopic rod acts here in a force-transmitting manner only in the direction of tensile force and is pushed together in the event of forces being applied externally to the pivot element.

In one embodiment, the force transmission element is rigid under pressure and is mounted movably in the direction of deformation of the spring element, as a result of which the cushioning effect and force-limiting effect when forces are applied externally can be achieved with passive displacement of the pivot element. In the event of active displacement of the pivot element by the drive, no deformation of the spring element takes place, and, in the event of passive displacement of the pivot element and a reversal of the direction of force, the force transmission element is displaced, for example rotated or moved.

The spring element can be in the form of a helical spring, spiral spring, disk spring, disk spring assembly or elastomer element; it is equally possible for the spring element to have a plurality of components which are constructed differently. The individual components can be elastic; equally, it is possible for the spring element to comprise a combination of elastic and inelastic components. A combination of helical spring, spiral spring and/or elastomer element is likewise possible in order to permit a return movement when a drive is reversed or a return movement after passive pivoting.

One embodiment of the invention makes provision for the force transmission element to be guided within the spring element in order to optimally use construction space. It is also possible to arrange a spiral spring or an elastomer element in the region of the articulated mounting of the pivot element independently of the positioning of the force transmission element, as a result of which any desired guidance of the force transmission element on the base and/or of the pivot element is made possible.

The drive is advantageously mounted on the base, preferably within the base, which can have a cavity for receiving a motor and optionally a gear mechanism, a control device, an energy store or the like. Alternatively, the drive is mounted in or on the force transmission element, in particular if the force transmission element is in the form of a hollow body, for example, a sleeve. As a result, the construction space which is taken up by the force transmission element can be optimally used.

The force transmission element can be mounted on the drive via a gear mechanism and/or a holder. The intermediate connection of a gear mechanism permits the use of small motors with a low power consumption at a high rotational speed, thus achieving advantages in terms of weight. It is possible by the selection of the gear mechanism to achieve the desired slow adjustment with a high degree of precision and at the same time large adjustment forces.

The pivot element is advantageously in the form of a prosthetic finger or in the form of a distal part of a prosthetic finger. The base can be in the form of a prosthetic hand chassis or proximal part of a prosthetic finger. The drive can comprise an electric motor and is advantageously self-locking such that, after a desired pivoting position is reached, no further energy has to be expended in order to hold the pivot element in the desired position.

The spring element is advantageously designed or arranged in such a manner that it is movable deformation together with the force transmission element out of the starting position into the pivoting position. This embodiment prevents energy from the drive or actuator being used for deforming the spring element, which would have a disadvantageous effect on the precision of the displacement movement, the speed and the entire energy to be used for displacing the pivot element. Without the deformation of the spring element during the active displacement into the pivoting position, more force can be applied for the closing movement and the maintaining of the closed position; furthermore, the energy store or rechargeable battery is better utilized.

### DESCRIPTION OF THE DRAWINGS

Exemplary embodiments of the invention will be explained in more detail below with reference to the attached figures, in which:

FIG. 1 shows a schematic illustration of the orthopedic device in the form of a prosthetic hand in an open position;

FIG. 2 shows the orthopedic device according to FIG. 1 during pivoting by the drive;

FIG. 3 shows an illustration of passive pivoting;

FIG. 4 shows a schematic illustration of a variant in an extension position;

FIG. 5 shows an illustration of FIG. 4 during passive flexion;

FIG. 6 shows an illustration of the orthopedic device after flexion via the drive;

FIG. 7 shows a sectional illustration through a prosthetic finger;

FIG. 8 shows an illustration of a detail from FIG. 7;

FIG. 9 shows a perspective illustration and an exploded illustration; and

FIG. 10 shows a variant of the invention with a force transmission element which is rigid under pressure.

### DETAILED DESCRIPTION

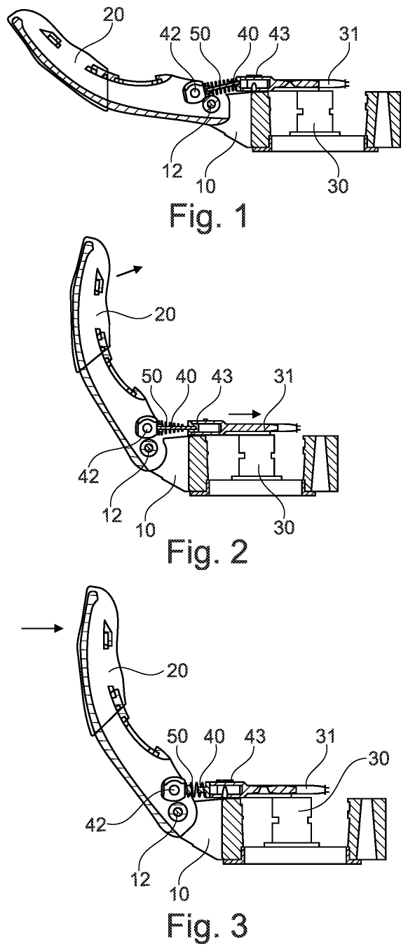

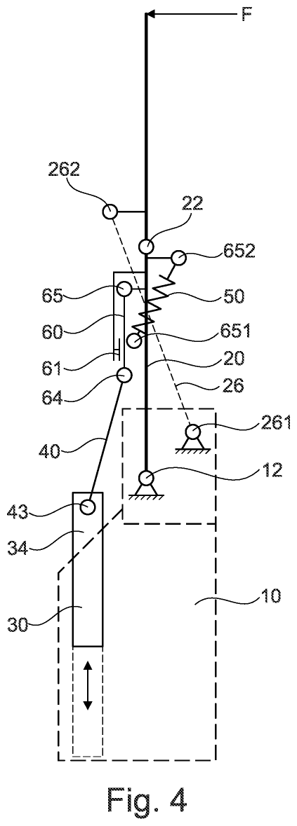

FIG. 1 shows a first variant of the invention, in which the orthopedic device is in the form of part of a prosthetic hand. A base 10 is illustrated in the form of a chassis of a prosthetic hand, on which chassis a prosthetic finger as a pivot element 20 is mounted pivotably about a pivot axis 12. The base 10 is arranged as a drive 30 in the form of an electric motor which, via a force transmission element 40, pivots the prosthetic finger 20 about the pivot axis 12 when the drive 30 is activated. In the exemplary embodiment which is illustrated, a carrier 31 with a receptacle and holder 43 for the force transmission element 40 is arranged on the electric motor. In the position of FIG. 4, the holder 43 is in an open starting position. The pivot element 20 or the prosthetic finger is in a maximally extended position. In the exemplary embodiment illustrated, the force transmission element 40 is in the form of a force transmission element 40 which is rigid under tension and yielding under pressure, for example in the form of a rope, cable, strap, chain or the like. A force transmission element 40 which is rigid under tension is understood as meaning in particular force transmission elements which, when the conventional forces are applied in orthopedic devices, carry out no, or a negligibly small, unintended lengthening.

A spring element 50 is arranged around the force transmission element 40, the spring element being in the form of a helical spring which is designed as a compression spring and, in the starting position, pretensioned the force transmission element 40 and thus exerts a compressive force on the bearing point of the finger-side holder 42. Both the finger-side holder 42 and the drive-side holder 43 can permit pivoting of the force transmission element 40 relative to the respective holder 42, 43. By means of the compressive force of the pretensioned spring 50, first of all the force transmission element 40 remains tensioned, and secondly, the pivot element 20 in the form of the prosthetic finger is held displaceably in the open starting position. The spring 50 is mounted between the two holders 42, 43 and is moved together with the force transmission element 40 when the drive 30 is activated and displacement of the force transmission element 40 by the drive 30 is brought about.

An active bending of the pivot element 20 relative to the base 10 is illustrated in FIG. 2. The drive 30 or actuator displaces the holder 43 to the right, as indicated by the arrow. The tensioned force transmission element 40 pulls to the right together with the holder 43 and causes a tensile force to be transmitted to the prosthetic finger 20 via the holder 42. Owing to the finger-side holder 42, which is positioned offset with respect to the pivot axis 12, a moment is exerted about the pivot axis 12 such that the pivot element 20 carries out a flexion movement and is displaced in the clockwise direction. During this active adjustment of the prosthetic finger 20, the spring 50 remains unchanged in respect of its length. The compression spring 50 is not compressed, and the entire driving energy of the drive 30 is introduced directly into the prosthetic finger 20 via the force transmission element 40. The force transmission element 40 in the form of a flexible rope remains tensioned throughout the entire pivoting movement without the spring element 50 being deformed.

FIG. 3 illustrates a passive flexion of the prosthetic finger 20. A compressive force, as indicated by the arrow, is applied to the prosthetic finger from the dorsal side, i.e. the rear side of the finger. The drive 30 is not activated, the drive-side holder 43 remains unchanged in the position which it also has in FIG. 1. Owing to the compressive force, the spring element 50 is compressed and deforms. The distance between the finger-side holder 42 and the drive-side holder 43 is reduced and the force transmission element 40 loses the tension. This is indicated by the wavy illustration of the force transmission element 40 in FIG. 3. In the case of an external force, which is not applied by the drive 30, in the flexion direction, the spring element 50 is therefore tensioned and the prosthetic finger 20 is displaced. As soon as the external force ceases, the spring element 50 is relaxed and the two holders 42, 43 are moved away from each other. The prosthetic finger 20 then pivots anticlockwise about the pivot axis 12 and moves again into the starting position according to FIG. 1. For the opening movement, no additional energy has to be applied by the drive 30. If after an active displacement, as has been described with reference to FIG. 2, a return movement is desired, the drive 30 is reversed, the drive-side holder 43 is moved to the left and a corresponding force is transmitted via the compression spring 50 to the prosthetic finger 20 such that pivoting about the pivot axis 12 takes place.

As an alternative to the yielding embodiment of the force transmission element 40 in the form of a rope or other flexible means, the force transmission element 40 can also be in the form of a movably mounted rod or a telescopic rod. Instead of a helical spring, the spring element 50 can also be in the form of a spiral spring, disk spring, disk spring stack or else in the form of a sleeve-shaped elastomer element. The arrangement of the force transmission element 40 within the spring element 50 permits a very compact construction and, in addition, protects the force transmission element 40 from external influences; basically, the force transmission element 40 and the spring element 50 can also be arranged next to each other separately from each other.

A variant of the invention which shows a schematic illustration of an orthopedic device in the form of a prosthetic hand with a multi-section prosthetic finger is illustrated in FIG. 4. The pivot element 20 shown on the base 10 is a prosthetic finger with a proximal pivot element 20 which is mounted on the base 10 so as to be pivotable about a proximal pivot axis 12. At the distal end of the proximal pivot element 20, a distal pivot element 25 is mounted pivotably on a distal pivot axis 22 in order, in an embodiment of a prosthetic finger, to provide a functionality which is approximate to that of a natural finger.

A drive 30 is arranged on the base 10, the drive preferably comprising an electric motor which moves a movable component 34 longitudinally, for example, via a rack drive or a spindle drive. Alternatively, displacement of the pivot element 20 can likewise be brought about via a rope or similar which is wound up. Depending on the direction of rotation of the motor, the movable component 34 is displaced in the one or other direction. The force transmission element 40 which, in the exemplary embodiment illustrated, is in the form of a rod which can also absorb compressive forces is fastened to the movable component 34. The force transmission element 40 is mounted pivotably at a proximal mounting point 43 on the movable component 34. At the opposite end, the force transmission element 40 is coupled at a distal mounting point 64 to the proximal pivot element 20 via a linkage mechanism 60. The linkage mechanism 60 has a parallel guide which is oriented substantially parallel to the longitudinal extent of the proximal pivot element 20. The linkage mechanism 60 is fastened via a lever to a distal mounting point 65 at a distance from the longitudinal extent of the proximal pivot element 20 such that, when a tensile force is applied to the force transmission element 40, a force component substantially parallel to the longitudinal extent of the proximal pivot element 20 is generated. Owing to the spaced mounting with respect to the connection between the two pivot axes 12, 22, this force component brings about a moment about the proximal pivot axis 12, and therefore the entire pivot element with the proximal pivot element 20 and the distal pivot element 25 is displaced anticlockwise. During a reverse movement, i.e. a movement of the movable component 34 upwards, a compressive force is exerted via the force transmission element 40 such that a reverse movement of the pivot element 20 takes place.

Furthermore, a spring element 50 in the form of a compression spring is arranged on the linkage mechanism 60, said spring element being mounted on the opposite side on the proximal pivot element 20 at a distance from the connecting line between the two pivot axes 12, 22. In the exemplary embodiment illustrated, the linkage mechanism 60 is arranged spaced apart on the volar or palmar side of the prosthetic finger, and the corresponding volar or palmar mounting point 651 of the spring element 50 is situated proximally from the distal mounting point 65. The second, dorsal mounting point 652 of the spring element 50 is situated on the opposite side on the proximal pivot element 20 proximally from the distal pivot axis 22. The spring element 50 presses the linkage mechanism 60 onto a bearing block 61 and holds it there via a pretensioning force.

In addition, on the base 10 in the manner spaced apart dorsally from the connecting line between the two pivot axes 12, 22, a connecting element 26 is fastened pivotably to the proximal pivot element 20 at a dorsal, proximal point of articulation 261. The second, distal point of articulation 262 is arranged on the distal pivot element 25 at a volar or palmar spacing from the connecting line between the two pivot axes 12, 22.

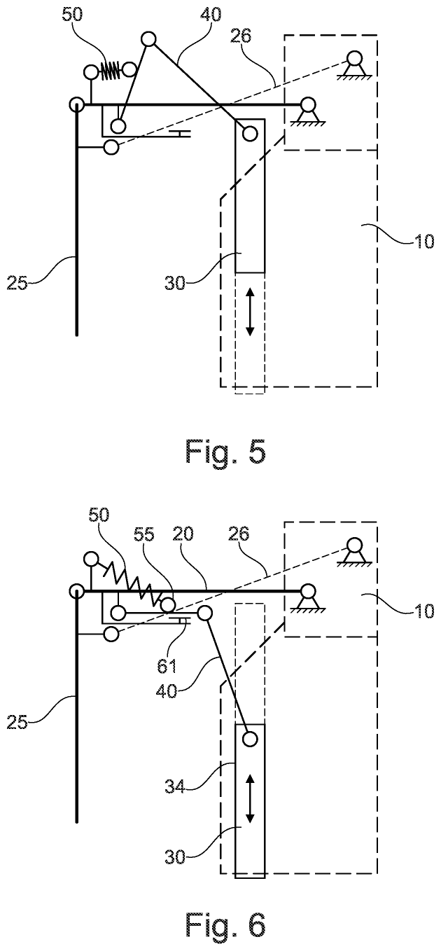

If an external force F is exerted on the distal pivot element 25 from the dorsal direction, a movement and a state, which is illustrated in FIG. 5, arise. The drive 30 with the movable element 34 is not activated and continues to be in the extended position, as is illustrated in FIG. 4 and also in FIG. 5. Owing to the exerted force F and the compressive force, exerted via the tensioned spring 50, on the linkage mechanism 60, a moment is produced anticlockwise about the proximal pivot axis 12. The proximal pivot element 20 is displaced anticlockwise, and the force transmission element 40 which is rigid under pressure pivots about the two mounting points 64, 65. The distal pivot element 25 pivots anticlockwise about the distal pivot axis 22 since the connecting element 26, as a component which is rigid under tension and is optionally flexible, generates a corresponding moment because of the spaced-apart points of articulation 261, 262. The spring element 50 is compressed.

If, by contrast, the drive 30 is activated, which is illustrated in FIG. 6, and the movable component 34 is moved downwards, the force transmission element 40 is correspondingly moved downwards. The spring element 50 is supported on the force transmission element 40 at an abutment 55 and is moved together therewith. The abutment 55 can be adjustable. A tensile force is exerted via the linkage mechanism 60 on the distal mounting point 65 such that a moment anticlockwise about the proximal pivot axis 12 is generated. The linkage mechanism 60 continues to remain mounted on the bearing block 61, and the spring element 50 together with the proximal pivot element 20 carries out a pivoting movement without being compressed. The distal pivot element 25 is coupled to the base 10 via the connecting element 26, and therefore a pivoting movement about the distal pivot axis 22 is brought about.

In an alternative embodiment, the compression spring 50 can also be replaced by a torsion spring which is arranged in the distal mounting point 65 and pretensions the linkage mechanism 60 in the direction of the bearing block 61.

It is possible, by means of the above-described arrangements of drive 30 and spring element 50, to keep the proximal pivot element 20 in a pretensioned position without said holding force counteracting an adjustment movement. As a result, the force which can be applied by the drive 30 as finger force, for example, can be fully used for the adjustment movement, and, for this purpose, the spring force of the spring element 50, the spring force pretensioning the pivot element 20 into the starting position, does not have to be overcome. As a result, the adjustment speed is increased while the energy to be used is simultaneously reduced.

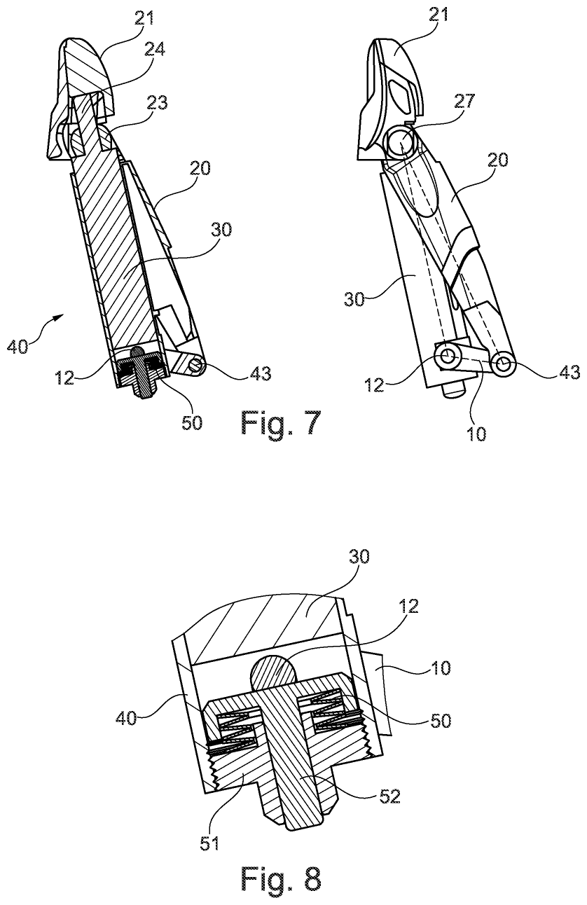

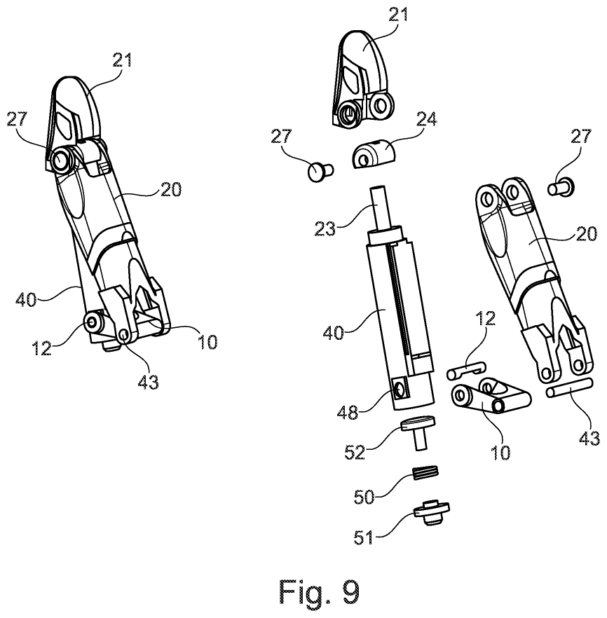

FIGS. 7 and 9 show variants of the invention with a drive 30 which is arranged within the force transmission element 40, here within a rear part of a prosthetic finger. FIG. 8 illustrates a view of a detail of the proximal mounting of the force transmission element 40 on the base 10 via a spring element 50. The force transmission element 40 is mounted on the base 10 so as to be movable and pivotable about a pin, acting as the pivot axis 12, in an elongated hole guide. A spring element 50 in the form of a disk spring assembly exerts a compressive force in the direction of the pivot axis 12. In the exemplary embodiment illustrated, the prosthetic finger is in the form of a prosthetic thumb and has a distal thumb part 21 which is mounted on the pivot element 20 so as to be pivotable about a distal pivot axis 27 which is formed by two half axes. The motorized drive 30 which drives a spindle 24 on which, in turn, a spindle nut 23 is mounted is located within the sleeve-shaped force transmission element 40. The force transmission element 40 forms a first proximal part of the thumb, the pivot element 20 forms the second proximal part of the thumb and is likewise mounted on the distal pivot axis 27. The distal thumb part 21 is used for gripping objects and is flexed when the force transmission element 40 is displaced relative to the pivot element 20. The pivot element 20 is used, for example, for gripping large objects when the object is in the region of a palm. The base 10 is a carrier via which the remaining components of the prosthetic finger are connected to the remainder of a prosthetic hand, for example to a chassis. The spindle nut 23 is mounted in a manner secure against rotation in the force transmission element 40, as a result of which, when the spindle 24 rotates, the spindle nut carries out a translational movement upwards and downwards and distally/proximally. As a result, the length ratios in the triangular joint between the pivot axis 12, an axis of articulation 43, at which the proximal end of the pivot element 20 is mounted pivotably on the carrier 10, and the distal pivot axis 27 change. In the event of a comparatively small change in length in the distance between the pivot axis 12 and the distal pivot axis 27, the prosthetic finger carries out a large pivoting angle about the axis of articulation 43 and the pivot axis 12. At the same time, only a small pivoting movement of the components about the distal pivot axis 27 is produced.

The pivot axis 12 as motor-side pivot axis is mounted in an elongated hole 48 under pretensioning in relation to the carrier or the base 10 via at least the spring element 50, which is in the form of an assembly of disk springs in the exemplary embodiment illustrated. In this case, the spring element 50 is supported in relation to the motor-side pivot axis 12 via a plunger 52 and is supported on the opposite side on the force transmission element 40 via a tensioning element 51. The force transmission element 40 has an internal thread in which an external thread on the tensioning element 51 engages. By screwing in or unscrewing the tensioning element 51, it is possible to change the spring pretensioning of the disk spring assembly 50 in the direction of the pivot axis 12. The elongated hole 48 in which the pivot axis 12 is guided in the form of a flattened pin is formed within the force transmission element 40. The plunger 52 is mounted within the flattened portion. It is possible via the elongated hole 48 to carry out a movement, which is directed in the axial direction of the force transmission element 40, counter to the spring pretensioning of the spring assembly 50. In the inoperative state, the spring assembly 50 has the effect that the axis 12 is pressed via the plunger 52 in the illustrated position onto the upper end of the elongated hole 48 within the force transmission element 40.

In the event of a passive load on the force transmission element 40 from the palmar side, i.e. in the event of a force exerted on the force transmission element 40 from the palm, a force which is directed in the proximal direction acts on the force transmission element 40 such that the housing or the force transmission element 40 with the elongated hole 48 is pressed strongly onto the axis 12. A change in the geometry and in the arrangement of the components of the prosthetic finger does not take place. If, by contrast, a force is exerted on the back of the thumb or a force, which acts in the dorsal direction, is exerted on the force transmission element 40, a force action component is produced in the distal direction, and therefore the pivot axis 12 is displaced along the elongated hole 48 counter to the spring force of the spring element 50. The pivot axis 12 is lifted out of the confines of the elongated hole 48 and presses the plunger 52 against the spring assembly 50, with the possible puff of movement depending on the magnitude of the introduced force and the spring pretensioning by the tensioning element 52. By means of the movement of the plunger 52, the springs of the spring element 50 are compressed since the tensioning element 51 is fixedly connected to the thread in the force transmission element 40 or in the thumb sleeve. The change in length in the distance between the distal pivot axis 27 and the pivot axis 12 permits flexion of the distal thumb part 21 and of the force transmission element 40 without the drive 30 having to be activated.

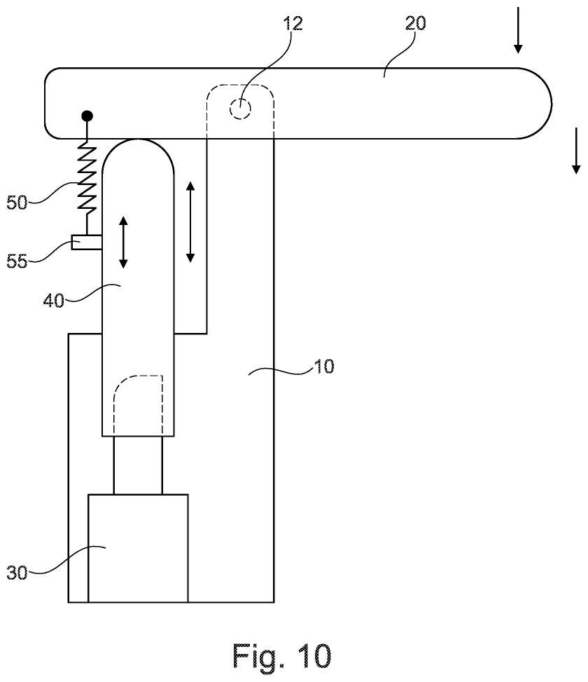

A further variant of the invention is illustrated schematically in FIG. 10. A drive 30 comprising an electric motor is fastened to the base 10. The force transmission element 40 is moved towards a proximal section of the pivot element 20 via, for example, a spindle drive or a screw sleeve, which is mounted so as to be non-rotatable and movable. As a result, a moment is exerted about the pivot axis 12, and therefore the pivot element 20 moves with its front end in the clockwise direction. The proximal section of the pivot element 20 is held in contact with the force transmission element 40 via the spring element 50, which is in the form of a pretensioned tension spring. If the motor is moved in the reverse direction and the drive 30 reversed, the force transmission element 40 retracts and a reverse pivoting movement about the pivoting axis 12 is carried out. Force is transmitted here via the spring element 50 which transmits the effective tensile forces. In the event of an external force F on a distal section of the pivot element 20, the pivot element 20 likewise pivots in the clockwise direction counter to the spring force which, by means of the spring element 50, is set against a pivoting movement. In the embodiment illustrated, the spring element 50 is mounted on an abutment 55 which can be displaced together with the force transmission element 40. The abutment 55 can be arranged adjustably on the force transmission element 40 in order to change the pretensioning of the spring element 50.

The advantage of the solution of the embodiments illustrated consists in that flexibility of the pivot element or prosthetic finger is provided in a certain load situation without, during normal operation, a passive displacement in the direction of flexibility having to overcome a resistance force. Return forces do not have to be overcome in order, when a drive is activated, to bring about a corresponding displacement of the components with respect to one another.

1. An orthopedic device, comprising:
a base;
a pivot element mounted in an articulated manner on the base;
a force transmission element for displacing the pivot element;
a drive connected to the force transmission element,
wherein the pivot element is displaceable from a starting position into a pivoting position that is pivoted in relation to the starting position,
wherein the force transmission element permits passive pivoting of the pivot element in a direction of the pivoting position without activation of the drive; and
a spring element designed for counteracting passive pivoting of the pivot element into the pivoting position assigned to the pivot element,
wherein the spring element is formed separately from the force transmission element, and
wherein the force transmission element blocks deformation of the spring element during pivoting by the drive into a respective pivoting position.

2. The orthopedic device according to claim 1, wherein the spring element is mounted on the force transmission element or on an abutment which is displaceable together with the force transmission element.

3. The orthopedic device according to claim 1, wherein the force transmission element is rigid under tension and yielding under pressure, wherein the force transmission element is in a form selected from the group consisting of a flexible tension means, a movably mounted rod, a movably mounted sleeve, and a telescopic rod.

4. The orthopedic device according to claim 1, wherein the force transmission element is rigid under pressure and is mounted movably in a direction of deformation of the spring element.

5. The orthopedic device according to claim 1, wherein the spring element is in a form selected from the group consisting of a helical spring, a spiral spring, a disk spring, and an elastomer element.

6. The orthopedic device according to claim 1, wherein the force transmission element is guided within the spring element.

7. The orthopedic device according to claim 1, wherein the drive is mounted on the base or on or in the force transmission element.

8. The orthopedic device according to claim 1 wherein the force transmission element is mounted on the drive via a gear mechanism and/or a holder.

9. The orthopedic device according to claim 1 wherein the pivot element is in a form of a prosthetic finger or in a form of a distal part of the prosthetic finger.

10. The orthopedic device according to claim 1, wherein the base is in a form selected from the group consisting of a prosthetic hand chassis, and proximal part of a prosthetic finger.

11. The orthopedic device according to claim 1, wherein the drive comprises an electric motor.

12. The orthopedic device according to claim 1 wherein the drive is self-locking.

13. The orthopedic device according to claim 1 wherein the spring element is movable without deformation with the force transmission element into the pivoting position.
